(19) United States
(12) Patent Application Publication
(10) Pub. No.: US 2004/0059001 A1
(43) Pub. Date: Mar. 25, 2004

(54) Extended release pharmaceutical composition containing metformin

(76) Inventors: Deepak Murpani, New Delhi (IN); Ashish Madan, New Delhi (IN); Vinod Kumar Arora, New Delhi (IN); Rajiv Malik, New Delhi (IN)

Correspondence Address: Jayadeep R Deshmukh, Ranbaxy Pharmaceuticals Inc, 600 College Road East, Suite 2100, Princeton, NJ, 08540, US

(21) Appl. No.: 10/432,654

(22) PCT Filed: Sep. 27, 2002
(86) PCT No.: PCT/IB02/03997

(30) Foreign Application Priority Data
Sep. 28, 2001 (IN) 1002/DEL/2001

(51) Int. Cl. A61K 31/16

(52) U.S. Cl. 514/630

(57) ABSTRACT

The present invention relates to an extended release pharmaceutical composition containing metformin and a rate controlling polymer and a process for the preparation thereof.

### FIELD OF INVENTION

The present invention relates to an extended release pharmaceutical composition containing metformin and a rate controlling polymer and a process for the preparation thereof.

### BACKGROUND OF THE INVENTION

Metformin is an oral antihyperglycemic agent of the biguanide class used in the management of non-insulin dependent diabetes mellitus (type 2-diabetes). Metformin hydrochloride is a highly water-soluble drug having poor flow and compressibility characteristics, hence, cannot be compressed in its pure form. Metformin is a high dose drug and therefore the tendency for capping is particularly high during the production of tablets. The capping results in losses of yield during the production and impairment of the quality.

U.S. Pat. No. 6,117,451 describes the use of specific excipients of particular size and density range to improve the flow and compressibility of metformin hydrochloride. These excipients are blended with metformin and the blend is then directly compressed. The use of excipients with a specific particle size and density range adds to the cost and makes the process tedious.

U.S. Pat. No. 5,955,106 describes a wet granulation process to prepare extended release metformin hydrochloride tablets. The process comprises of granulating metformin and a hydrocolloid forming retarding agent with an aqueous solvent to form a granulated product and drying the granulated product to a residual moisture content of about 0.5 to 3% by weight.

WO 99/47128 describes a method for preparing a biphasic controlled release metformin tablets. The method comprises forming an inner solid particulate phase in the form of individual particles containing metformin and an extended release material and mixing the individual particles forming the inner solid particulate phase with an outer solid continuous phase comprising an extended release material in which the particles of inner solid particulate phase are dispersed and embedded. The inner particulate phase is prepared by wet granulation of metformin and the extended release material with water or organic solvents. The inner particulate phase is then dried and mixed with the outer continuous phase and compressed to form tablets.

The wet granulation process when used to agglomerate the powder mix provides adequate flow characteristics to the controlled release matrix formulation. But most hydrophilic polymers often interact with the aqueous system making wet granulation difficult. The wet granulation process may also result in variable release characteristics depending on the degree of hydration of the polymer. Even the fluid volume of the granulating agent and granulation time may also affect the release characteristics. The use of organic solvent leads to the problem of residual solvents.

It is, therefore, desirable to provide a simple process of production which does not require wet granulation with organic solvents or water, and the use of specific and expensive directly compressible excipients, but imparts good flow and compressibility characteristics to the blend, solves the problem of capping, and provides the desired extended release.

### SUMMARY OF THE INVENTION

The above mentioned object is achieved by a unique granulation/densification process of the present invention. Conventionally, a dry granulation process is carried out by compaction or slugging of the blend without the aid of moisture. The inherent moisture of the drug, or the excipients, or the applied compaction force provides cohesiveness and binding between the particles. In the present invention, it has been surprisingly found that increase in water content of the granules, not only eliminates the capping problem but also imparts better hardness, elegance to the tablet, and reduces the friability considerably.

It was also found that the granules having water content 3.2% w/w gave acceptable tablets but increased water content up to 8% w/w provided better hardness (up to 1½ times more), improved elegance and reduces friability up to ¼. The water content of granules could be increased up to 10%; however the increase above 7% did not improve the physical characteristics further. Interestingly, the residual water content of the tablet did not increase exponentially with the increase in water content of the blend. Even the addition of 10% water produced tablets with 5% final water content.

The desired water content of the granules was obtained by moisture conditioning. The moisture conditioning was done by:

the addition of limited quantity of water to the ingredient(s)/blend; or

exposing the ingredient(s) or blend to higher humidity conditions; or

choosing such excipients, which provide optimum water content to the blend.

### DETAILED DESCRIPTION OF THE INVENTION

The present invention relates to an extended release pharmaceutical composition comprising metformin and a rate controlling polymer, wherein the pharmaceutical composition has a water content from about 3.2 to about 10.0% by weight.

The present invention further relates to a process for producing an extended release metformin pharmaceutical composition comprising the step of moisture conditioning of metformin alone or its blend with a rate controlling polymer and pharmaceutically acceptable excipients, wherein the pharmaceutical composition has a water content from about 3.2% to about 10.0% by weight.

The term moisture conditioning means imparting optimum water content to granules. The optimum water content of the granules for the purpose of the present invention is about 3.2% to 10.0% w/w.

The process of the present invention is carried out by

(a) moisture conditioning of the ingredient(s), which may include metformin, rate controlling polymers and other pharmaceutically acceptable excipient(s) or a blend of two or more ingredients;

(b) compacting or slugging;

(c) milling or crushing the compacted/slugged material of step (b) into granules; and

(d) compressing the granules to form tablets.

The granules produced by the process of the present invention are simple to manufacture, have good flow characteristics and are easy to compress even on industrial scale. The process of the present invention gives good yield as losses due to capping are completely avoided.

Moreover, as the present process differs from the wet granulation process in employing the limited amount of water, it eliminates the variability in the degree of hydration of hydrophilic polymers and release characteristics. Even the variability in fine to coarse ratio of granules does not affect the release characteristics. The process provides desired granules and fines in one cycle, unlike conventional dry granulation process, thereby reducing the process time. It also reduces the dust generation, which is a common problem with the dry granulation process. The process has good reprocessing potential as the compacts/slugs/tablets can be crushed into powder and re-compacted to make the tablets without change in release profiles.

Therefore, the present invention provides a process for producing the metformin extended release tablets that have better strength, yield, aesthetic appeal and desired release profile. The process is particularly useful for eliminating the capping problem.

For the present invention, metformin can be used in the form of acid addition salts of inorganic or organic acids. These acids are exemplified by, but in no way limited to, acids such as hydrochloric acid, formic acid, acetic acid, malic acid, tartaric acid or fumaric acid. The hydrochloric salt is preferably used.

The rate controlling polymers can be selected from any such pharmaceutically acceptable excipients, which can control the rate of release of the active ingredient. Preferably such rate-controlling polymer are selected from the group consisting of cellulose derivatives, starch, gums, alginates, acrylic acid derivatives and carbohydrate based polymers.

The cellulose derivative are selected from the group consisting of ethyl cellulose, methylcellulose, hydroxymethylcellulose, hydroxyethylcellulose, hydroxypropylcellulose, hydroxypropyl methylcellulose and sodium carboxymethylcellulose of different degree of substitution and molecular weights. These rate-controlling polymers can be used alone or in combination. Various degrees of substitution and/or different molecular weights corresponding to a different degree of viscosity can be used as suitable cellulose based rate-controlling polymers.

The rate controlling polymer can be used in a concentration of 10% to 60% depending on the polymer used. The use of hydroxypropyl methylcellulose (HPMC), hydroxypropylcellulose, carboxymethylcellulose is preferred. These polymers swell to form a hydrophilic matrix system, which control the release of metformin hydrochloride. The tablet hydrates on wetting and hydrophilic polymers forms a gel layer. Due to water permeation into the tablet, the thickness of gel layer is increased and the metformin hydrochloride diffuses slowly out of the gel layer.

The pharmaceutically acceptable excipients of the invention may be selected from amongst the diluent, binder, disintegrants, lubricants, glidants, coloring agents and flavoring agents which are chemically and physically compatible with metformin and which would help in optimizing tablet hardness, friability and drug dissolution.

The diluents of this invention may be selected from any such pharmaceutically acceptable excipients, which gives bulk to the composition and improves the compressibility. The diluents are selected from starch, microcrystalline cellulose, lactose, glucose, mannitol, alginates, alkali earth metal salts, clays or polyethylene glycols. Microcrystalline cellulose is particularly preferred as it has better compressibility.

The binders of this invention may be selected from any such pharmaceutically acceptable excipients, which has cohesive properties to act as a binder. The binders are selected from the group consisting of starch, microcrystalline cellulose, highly dispersed silica, mannitol, lactose, polyethylene glycol, polyvinylpyrrolidone, cross-linked polyvinylpyrrolidone, cross-linked carboxymethylcellulose, hydroxypropyl methyl cellulose and hydroxypropylcellulose and natural and synthetic gums.

The disintegrants for the present invention may be selected from starches or modified starches such as sodium starch glycolate, corn starch, potato starch or pregelatinized starch, clays such as bentonite, montmorillonite or veegum; celluloses such as microcrystalline cellulose, hydroxypropyl cellulose or carboxymethylcellulose, algins such as sodium alginate or alginic acid; cross-linked cellulose such as croscarmellose sodium, gums such as guar gum or xanthan gum; cross-linked polymers such as crospovidone; effervescent agents such as sodium bicarbonate and citric acid; or mixtures thereof.

The lubricants of the present invention may be selected from talc, magnesium stearate, and other alkali earth metal stearate like calcium, zinc etc., lauryl sulphate, hydrogenated vegetable oil, sodium benzoate, sodium stearyl fumarate, glyceryl monostearate and PEG 4000.

The glidants of the present invention may be selected from colloidal silicon dioxide and talc.

For the purpose of the present invention, the moisture conditioning is done by the addition of limited quantity of water to the ingredient(s)/blend or exposing the ingredient(s) or blend to higher humidity conditions; or choosing such excipients, which provide optimum water content to the blend. Generally, the moisture conditioning of any ingredient can be done however the moisture conditioning of rate controlling polymer should be avoided.

For moisture conditioning by the addition of water, a desired amount of water is added slowly to the ingredient/blend with a constant mixing in a suitable mixer to avoid lump formation and maintain free flowing characteristics of the blend/ingredients.

However, for moisture conditioning by humidity, either the ingredient/blend is kept in trays and exposed to a relative humidity of more than 50% or complete process is carried out under elevated humidity conditions (i.e. more than 50% relative humidity).

The moisture conditioned blend or blend containing moisture-conditioned ingredients is compacted by roller compaction. The compactor can be concave or convex, having straight or proliferated roller or different design of powder transport screws. Alternatively, this blend can be compressed to make slugs. For the purpose of the present invention the compaction or slugging, can be done of either metformin alone or with a rate controlled polymer and/or with excipient(s);

The compacted/slugged material is crushed/milled by a suitable milling machine like oscillating granulator/Multimill/Fitzmill and sieved into the desired granule size.

As an optional step, the granules that are either too large or too small are recycled and combined with original powder mix and passed through a roller compactor or a tabletting machine. Normally 30-70% of coarse granules (retained on 60-mesh sieve) is preferred and is usually achieved in a single compaction cycle.

These granules are lubricated with the lubricant and are compressed into tablets. Optionally, these granules can be capsulated into hard gelatin capsules.

Alternatively, moisture conditioning can be done by mixing the moisture-conditioned ingredient to granules produced by compaction or slugging or by exposing these granules to higher humidity or carrying out the process under elevated humidity conditions.

The invention is further illustrated by, but is by no-means limited to, the following examples.

In the following example 1, the tablets were prepared by conventional dry granulation process. However in comparative examples 2A-2C, the water content of the blend was adjusted to produce granules having water content between about 3.2 to 8% w/w.

### EXAMPLE 1

### Tabl ts pr pared by conventi nal dry granulati n t chnique

| Ingredi nt | Weight (mg) per tablet |
|---|---|
| Metformin hydrochloride | 500.00 |
| Sodium carboxymethylcellulose | 36.00 |
| Microcrystalline cellulose | 60.00 |
| Hydroxypropyl methyl cellulose | 398.00 |
| Magnesium stearate | 6 |
| Water | q.s. |
| Total | 1000.00 |

Water content (determined by Karl Fischer apparatus) of the granules −2.8% w/w

Process:

1. Metformin Hydrochloride, Microcrystalline cellulose, Hydroxypropyl methylcellulose and sodium carboxymethylcellulose are sifted through 40 BSS sieve and lubricated with magnesium stearate.

2. Blend of step 1 is compacted.

3. Compacts are sized through oscillating granulator and sifted through 18 BSS sieve.

4. Fines obtained are recycled to achieve desired ratio of coarse and fines.

5. Granules of step 4 are lubricated with magnesium stearate and compressed.

### COMPARATIVE EXAMPLE 2A-2C

### Tablets prepared by the granulation process of the pr sent inv ntion comprising the moisture-conditioning st p

| | Weight (mg) per tablet | | |
|---|---|---|---|
| Ingredient | 2A | 2B | 2C |
| Metformin hydrochloride | 500.00 | 500.00 | 500.00 |
| Sodium carboxymethylcellulose | 36 | 36 | 36 |
| Microcrystalline cellulose | 60 | 60 | 60 |
| Hydroxypropyl methyl cellulose | 365.5 | 348.0 | 298.0 |
| Magnesium stearate | 6 | 6 | 6 |
| Water | q.s. | q.s. | q.s |
| Total | 1000 | 1000 | 1000 |
| Water content of the granules (% w/w) | 3.77 | 4.71 | 6.20 |

Process:

1. The ingredients are weighed and sifted through 40BSS sieve.

2. Metformin hydrochloride and microcrystalline cellulose are mixed in a blender and water is added slowly with mixing. The mixture is passed through the sieve and mixed with other ingredients in the blender. Subsequently, magnesium stearate (half of the quantity) is screened through a 60-mesh screen, added to the blender, and mixed for 5 minutes.

3. The resultant final blend is compacted using the roller compactor.

4. Compacted material is milled and screened through 18-mesh screen.

5. The granules so obtained are lubricated with magnesium stearate using a twin shell blender (rest of the quantity).

6. And compressed on a 16-station tabletting machine to tablets with an average weight of 1 gm.

The physical properties of the tablets prepared by conventional dry granulation technique as per the composition of Example 1 and by granulation process of the present invention comprising the moisture conditioning step as per examples 2A, 2B and 2C were evaluated using the following parameters.

(i) Mean Hardness—The hardness of each tablet was measured with a Schleuniger Hardness Tester. The test was performed on 6 tablets and the mean calculated.

(ii) Friability—Friability testing was done as per the method described in USP -24/NF-19, 2000, Page 2148-49.

(iii) Elegance—The elegance was evaluated by physical appearance of the tablets (smoothness and gloss), at initial and after 30 days storage in HDPE bottles under controlled conditions of 40° C./75% RH.

Further, the release profiles of the tablets prepared by the above examples/comparative examples were compared in pH 6.8 phosphate buffer/900 ml/100 rpm/USP 1 apparatus/233 nm. The results are summarized in Table 1 and Table. 2.

TABLE 1
Physical properties of Metformin hydrochloride tablets prepared as
per the composition of Example 1 and Comparative Examples 2A-2C.

| | | Comparative Examples | | |
|---|---|---|---|---|
| Physical properties | Example 1 | 2A | 2B | 20 |
| Hardness (Kp) | 16.9 | 20.85 | 23.50 | 23.15 |
| Friability (% w/w) | 0.43 | 0.10 | 0.099 | 0.10 |
| Elegance | + | ++ | +++ | +++ |
| Elegance after 30 days | − | + | +++ | +++ |
| storage at 40° C./75% RH | | | | |

− Rough tablet surface
+, ++, +++ Denote the degree of elegance

TABLE 2
% R lease Profile of M tformin hydrochloride tabl ts pr pared as p r
the comp sition of Examples-1 and Comparative Examples—2A-2C
at pH 6.8 phosphat buffer/900 ml/100 rpm/USP 1 apparatus/233 nm.

| | | Drug release % | | |
|---|---|---|---|---|
| | | Comparative Examples | | |
| Time (hr) | Example1 | 2A | 2B | 2C |
| 1 | 27.1% | 32.0 | 32.0 | 32.4 |
| 4 | 58.7% | 64.0 | 64.0 | 66.1 |
| 8 | 84.9% | 91.0 | 89.0 | 89.4 |
| 12 | 97.8% | 102.0 | 102.0 | 97.0 |

While the present invention has been described in terms of its specific embodiments, certain modifications and equivalents will be apparent to those skilled in the art and are intended to be included within the scope of the present invention.

We claim:

1. An extended release pharmaceutical composition comprising metformin and a rate controlling polymer, wherein the pharmaceutical composition has a water content from 3.2 to about 10.0% by weight.

2. The pharmaceutical composition according to claim 1 wherein the water content is from 3.2 to about 8.0% by weight.

3. The pharmaceutical composition according to claim 2 wherein the water content is from 3.5 to about 6.0% by weight.

4. The pharmaceutical composition according to claim 1, 2 or 3 wherein the water content is obtained by moisture conditioning.

5. The pharmaceutical composition according to claim 4 wherein the moisture conditioning is done by the addition of water or by exposing to higher humidity or by choosing excipients having high water content.

6. The pharmaceutical composition according to claim 5 wherein the moisture conditioning is done by the addition of water.

7. The pharmaceutical composition according to claim 5 wherein the moisture conditioning is done by exposing to higher humidity.

8. The pharmaceutical composition according to claim 5 wherein the moisture conditioning is done by choosing the excipients having high water content.

9. The pharmaceutical composition according to claim 1 wherein the metformin is used in the form of acid addition salts of inorganic or organic acid.

10. The pharmaceutical composition according to clam 10 wherein the metformin is used as a hydrochloride salt.

11. The pharmaceutical composition according to claim 1 wherein the rate controlling polymer is selected from the group consisting of cellulose derivatives, starch, gums, alginates, acrylic acid derivatives and carbohydrate based polymers.

12. The pharmaceutical composition according to claim 11 wherein the rate controlling polymer is a cellulose derivative.

13. The pharmaceutical composition according to claim 12 wherein the cellulose derivative is selected from the group consisting of ethyl cellulose, methylcellulose, hydroxymethylcellulose, hydroxyethylcellulose, hydroxypropylcellulose, hydroxypropyl methylcellulose, sodium carboxymethylcellulose, and a mixture thereof.

14. The pharmaceutical composition according to claim 13 wherein the cellulose derivative is hydroxypropyl methylcellulose.

15. The pharmaceutical composition according to claim 13 wherein the cellulose derivative is carboxymethylcellulose.

16. The pharmaceutical composition according to claim 13 wherein the cellulose derivative is a combination of hydroxypropyl methylcellulose and carboxymethylcellulose.

17. The pharmaceutical composition according to claim 1 wherein it further comprises other pharmaceutically acceptable excipients.

18. The pharmaceutical composition according to claim 17 wherein the other pharmaceutically acceptable excipients are selected from diluents, binders, disintegrants, lubricants, glidants, coloring agents and flavoring agents.

19. The pharmaceutical composition according to claim 18 wherein the diluent is selected from the group consisting of starch, microcrystalline cellulose, lactose, glucose, mannitol, alginates, alkali earth metal salts, clays or polyethylene glycols.

20. The pharmaceutical composition according to claim 19 wherein the diluent is microcrystalline cellulose.

21. The pharmaceutical composition according to claim 1 wherein the composition is in the form of granules, tablets or capsules.

22. A process for producing an extended release metformin pharmaceutical composition, comprising moisture conditioning of metformin, wherein the pharmaceutical composition has a water content from 3.2% to about 10.0% by weight.

23. The process of claim 22 further comprising blending moisture conditioned metformin with a rate controlling polymer and other pharmaceutically acceptable excipient(s).

24. The process according to claim 22 wherein the process comprises moisture conditioning of a blend of metformin and other pharmaceutically acceptable excipients.

25. The process of claim 24 comprising blending moisture conditioned metformin and other pharmaceutically acceptable excipients with a rate controlling polymer.

26. The process according to claim 22 wherein the process comprises moisture conditioning of a blend of metformin, rate controlling polymer and other pharmaceutically acceptable excipients.

27. The process according to claim 23, 25 or 26 wherein the blend is further subjected to compaction or slugging.

28. The process according to claim 27 wherein the blend is compacted.

29. The process according to claim 28 wherein the compaction is done by roller compaction.

30. The process according to claim 27 wherein the blend is slugged.

31. The process according to claim 27 wherein the compacted or slugged material is further milled or crushed into granules.

32. The process according to claim 31 wherein the compacted or slugged material is milled to produce granules.

33. The process according to claim 31 wherein the compacted or slugged material is crushed to produce granules.

34. The process according to claim 31 wherein the granules are further compressed into tablets.

35. The process according to claim 34 wherein the granules are lubricated before compression.

36. The process according to claim 23, 25 or 26 wherein the pharmaceutically acceptable excipient is selected from diluents, binders, disintegrants, lubricants, glidants, coloring agents and flavoring agents.

37. The process according to claim 36 wherein the pharmaceutically acceptable excipient is a diluent.

38. The process according to claim 37 wherein the diluent is selected from the group consisting of starch, microcrystalline cellulose, lactose, glucose, mannitol, alginates, alkali earth metal salts, clays or polyethylene glycols.

39. The process according to claim 38 wherein the diluent is microcrystalline cellulose.

40. The process according to claim 34 wherein the tablets have improved hardness, elegance and reduced friability.
